(19) United States
(12) Patent Application Publication
(10) Pub. No.: US 2008/0051406 A1
(43) Pub. Date: Feb. 28, 2008

(54) BRIMONIDINE AND TIMOLOL COMPOSITIONS

(76) Inventors: Richard Graham, Irvine, CA (US); Rhett Schiffman, Laguna Beach, CA (US)

Correspondence Address: ALLERGAN, INC., 2525 DUPONT DRIVE, T2-7H, IRVINE, CA, 92612-1599, US

(21) Appl. No.: 11/843,294

(22) Filed: Aug. 22, 2007

Related U.S. Application Data
(60) Provisional application No. 60/823,523, filed on Aug. 25, 2006.

(51) Int. Cl. A61K 31/502; A61P 27/00; A61P 27/06

(52) U.S. Cl. 514/249

(57) ABSTRACT

Disclosed herein are compositions containing brimonidine and timolol. Therapeutic methods and medicaments related thereto are also disclosed.

### RELATED APPLICATION

This application claims the benefit of U.S. Provisional Application Ser. No. 60/823,523, filed Aug. 25, 2006, which is hereby incorporated by reference in its entirety.

### DESCRIPTION OF THE INVENTION

Ocular hypotensive agents are useful in the treatment of a number of various ocular hypertensive conditions, such as post-surgical and post-laser trabeculectomy ocular hypertensive episodes, glaucoma, and as presurgical adjuncts.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. In cases where surgery is not indicated, topical β-adrenoreceptor antagonists have traditionally been the drugs of choice for treating glaucoma.

Disclosed herein is a composition comprising brimonidine having a concentration from about 1 mM to about 4.5 mM timolol having a concentration from about 2 mM to about 15.8 mM, and benzalkonium chloride having a concentration of from about 150 to 250 ppm.

These compositions are useful for treating glaucoma or reducing intraocular pressure.

Also disclosed is a medicament for the treatment of glaucoma or ocular hypertension by topical administration to an eye of a mammal, said medicament comprising brimonidine having a concentration from about 1 mM to about 4.5 mM timolol having a concentration from about 2 mM to about 15.8 mM, and benzalkonium chloride having a concentration of from about 150 to 250 ppm.

Also disclosed is a method comprising topically administering a composition to an eye of a mammal, said method being useful for the treatment of glaucoma or ocular hypertension, wherein said composition comprises brimonidine having a concentration from about 1 mM to about 4.5 mM timolol having a concentration from about 2 mM to about 15.8 mM, and benzalkonium chloride having a concentration of from about 150 to 250 ppm.

Also disclosed is use of a composition in the manufacture of a medicament for the treatment of glaucoma or ocular hypertension, said composition comprising brimonidine having a concentration from about 1 mM to about 4.5 mM timolol having a concentration from about 2 mM to about 15.8 mM, and benzalkonium chloride having a concentration of from about 150 to 250 ppm.

Brimonidine is a compound having the formula shown below. It is commercially available from Allergan, Inc. for the treatment of glaucoma or ocular hypertension in the form of the tartrate salt as Alphagan® (0.2% brimonidine tartrate) or Alphagan P® (0.15% brimonidine tartrate). However, for the purposes of this disclosure, “brimonidine” refers to any salt of brimonidine, not just the tartrate, as well as the free base.

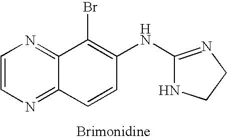

One composition comprises brimonidine tartrate.

In another composition the concentration of brimonidine is from about 2 mM to about 3.5 mM.

In another composition the concentration of brimonidine is about 3.4 mM.

In another composition the concentration of brimonidine is about 2.26 mM.

Timolol is a compound having the formula shown below. It is commercially available from a number of sources, generally in the form of a solution a concentration of 0.5% or 0.25% of the maleate salt. However, for the purposes of this disclosure, “timolol” refers to any salt of timolol, not just the maleate, as well as the free base.

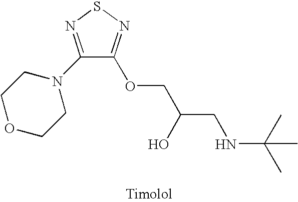

In one composition, the concentration of timolol is about 15.8 mM or about 7.9 MM.

In another composition, the concentration of timolol is from about 5 mM to 15.8 about mM.

The abbreviation “mM” refers to millimolar concentration, i.e. 10⁻³ M, as generally recognized in the art. For a liquid which is not a homogenous liquid, such as an emulsion, the millimolar concentration is taken to include the number of millimoles of the compound divided by the volume of the liquid in liters. The volume of the liquid is the volume of the entire liquid, including all oil and water phases.

In certain compositions the concentration of BAK is from 150 ppm to 200 ppm. In other compositions the concentration of BAK is from 150 ppm to 200 ppm. In other compositions the concentration of BAK is from 150 ppm to 250 ppm.

A liquid which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions are often maintained at a comfortable pH with an appropriate buffer system. The formulations may also contain conventional, pharmaceutically acceptable preservatives, stabilizers and surfactants.

Various buffers and means for adjusting pH may be used so long as the resulting preparation is ophthalmically acceptable. Accordingly, buffers include, but are not limited to, acetate buffers, citrate buffers, phosphate buffers and borate buffers. Acids or bases may be used to adjust the pH of these formulations as needed.

In many compositions, a pH of from about 5.5 to about 9 is desirable. In other compositions, the pH is from about 6 to about 8. In other compositions, the pH is from about 7.4 to about 8.5.

Certain compositions contain solubility enhancing components (SECs) in amounts effective to enhance the solubility of brimonidine at a given pH. These SECs may be anionic in nature, and can be polymeric in nature. In one embodiment the SEC is a cellulose derivative, in another embodiment the SEC is not a cellulose derivative or a cyclodextrin. In these compositions, the SEC is used to enhance the solubility of brimonidine. In other words, in two compositions containing brimonidine which are identical except for the presence of an effective amount of the SEC, more brimonidine will be dissolved in the composition containing the SEC than the in the composition not containing the SEC.

The SEC may include a non-ionic or polyanionic component. As used herein, the term “polyanionic component” refers to a chemical entity, for example, an ionically charged species, such as an ionically charged polymeric material, which includes multiple discrete anionic charges. Non-ionic SECs may include polyvinyl alcohol (PVA), polyvinyl pyrrolidone (povidone), and various gums and other non-ionic agents.

In one embodiment, the SEC is a polyanionic component, which may be selected from polymeric materials having multiple anionic charges, and mixtures thereof.

Examples of useful polyanionic components are selected from anionic polymers derived from acrylic acid (meaning to include polymers from acrylic acid, acrylates and the like and mixtures thereof), anionic polymers derived from methacrylic acid (meaning to include polymers from methacrylic acid, methacrylates, and the like and mixtures thereof), anionic polymers derived from alginic acid (meaning to include alginic acid, alginates, and the like and mixtures thereof), anionic polymers of amino acids (meaning to include polymers of amino acids, amino acid salts, and the like and mixtures thereof), and the like, and mixtures thereof. Very useful polyanionic components are those selected from anionic cellulose derivatives and mixtures thereof, especially carboxymethyl cellulose and its derivatives.

A surfactant may be used for assisting in dissolving an excipient or an active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phosphalipids, phosphatidyl chloline, phosphatidyl serine, and the like.

Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium (EDTA), although other chelating agents may also be used in place or in conjunction with it.

Certain compositions comprise an effective amount of EDTA. In other compositions, concentration of EDTA is at least 0.001%. In other compositions, the concentration of EDTA is at least 0.01%. In other compositions the concentration of EDTAis 0.15% or less. In other compositions the concentration of EDTA is 0.1% or less. In other compositions the concentration of EDTA is 0.05% or less.

Compositions may be aqueous solutions or emulsions, or some other acceptable liquid form. For an emulsion, one or more oils will be used to form the emulsion, and in some instances one or more surfactants and/or emulsion stabilization excipients will be required. Suitable oils include, but are not limited to anise oil, castor oil, clove oil, cassia oil, cinnamon oil, almond oil, corn oil, arachis oil, cottonseed oil, safflower oil, maize oil, linseed oil, rapeseed oil, soybean oil, olive oil, caraway oil, rosemary oil, peanut oil, peppermint oil, sunflower oil, eucalpytus oil, sesame oil, and the like.

### EXAMPLE 1

Aqueous solution compositions may be prepared according to Table 1.

TABLE 1

| | Formulation | | | | | |
|---|---|---|---|---|---|---|
| | 1 | 2 | 3 | 4 | 5 | 6 |
| Brimonidine Tartrate (mM) | 3.39 | 3.39 | 2.26 | 2.26 | 2 | 2 |
| Timolol Maleate, EP (mM) | 15.8 | 15.8 | 15.8 | 7.9 | 7.9 | 7.9 |
| Benzalkonium Chloride, NF, | 150 | 150 | 200 | 200 | 200 | 200 |
| EP (ppm) | | | | | | |
| Sodium Phosphate, monobasic | 0.43 | 0.43 | 0.43 | 0.43 | 0.43 | 0.43 |
| monohydrate, USP (% w/v) | | | | | | |
| Sodium Phosphate, dibasic | 2.15 | 2.15 | 2.15 | 2.15 | 2.15 | 2.15 |
| heptahydrate, USP (% w/v) | | | | | | |
| Sodium Hydroxide, NF | 6.9 | 7.4 | 7.8 | 7.8 | 7.8 | 7.8 |
| (adjust to pH shown) | | | | | | |
| Hydrochloric Acid, NF (adjust | 6.9 | 7.4 | 7.8 | 7.8 | 7.8 | 7.8 |
| to pH shown) | | | | | | |
| Carboxymethylcellulose | — | 0.5 | 1 | 1.5 | 1.5 | 1.5 |
| (% w/v) | | | | | | |
| Purified Water, USP, EP | q.s. | q.s. | q.s. | q.s. | q.s. | q.s. |
| | ad | ad | ad | ad | ad | ad |

### EXAMPLE 2

Emulsions are formulated with the compositions shown in Table 2 using the method described in U.S. Pat. No. 5,981,607, incorporated herein by reference, with the brimonidine and timolol being added to the castor oil before introducing the oil into the emulsion.

| | Formulation | | |
|---|---|---|---|
| | 1 | 2 | 3 |
| Brimonidine Tartrate (mM) | 3.39 | 2.26 | 2.26 |
| Timolol Maleate, EP (mM) | 15.8 | 15.8 | 7.9 |
| Castor Oil (% w/w) | 1.25 | 1.25 | 1.25 |
| Polysorbate 80 (% w/w) | 1.0 | 1.0 | 1.0 |
| Pemulen ® (% w/w) | 0.1 | 0.1 | 0.1 |
| Glycerin (% w/w) | 1.0 | 1.0 | 1.0 |
| Boric Acid (% w/w) | 0.6 | 0.6 | 0.6 |
| Benzalkonium Chloride, NF, EP (ppm) | 150 | 150 | 200 |
| Purified Water | qs. ad. | qs. ad. | qs. ad. 100 |
| | 100 | 100 | |

### EXAMPLE 3

A patient suffering from elevated intraocular pressure or glaucoma is treated with a composition of Example 1 or 2. The composition is administered topically to the eyes of the patient twice a day. Within a few hours reduction in pressure is observed, and an acceptable pressure is achieved within one or two days. Normal intraocular pressure is maintained for as long as the patient receives the composition twice a day.

What is claimed is:

1. A composition comprising brimonidine having a concentration from about 1 mM to about 4.5 mM timolol having a concentration from about 2 mM to about 15.8 mM, and benzalkonium chloride having a concentration of from about 150 to 250 ppm.

2. The composition of claim 1 wherein the concentration of timolol is about 15.8 mM or 7.9 mM.

3. The composition of claim 2 wherein the concentration of brimonidine is about 3.4 mM.

4. The composition of claim 2 wherein the concentration of brimonidine is about 2.26 MM.

5. The composition of claim 1 wherein the pH is from about 7.4 to about 8.5.

6. The composition of claim 5 which further comprises a solubility enhancing component.

7. The composition of claim 1 which further comprises EDTA.

8. A medicament for the treatment of glaucoma or ocular hypertension by topical administration to an eye of a mammal, said medicament comprising brimonidine having a concentration from about 1 mM to about 4.5 mM timolol having a concentration from about 2 mM to about 15.8 mM, and benzalkonium chloride having a concentration of from about 150 to 250 ppm.

9. A method comprising topically administering a composition to an eye of a mammal, said method being useful for the treatment of glaucoma or ocular hypertension, wherein said composition comprises brimonidine having a concentration from about 1 mM to about 4.5 mM timolol having a concentration from about 2 mM to about 15.8 mM, and benzalkonium chloride having a concentration of from about 150 to 250 ppm.

10. Use of a composition in the manufacture of a medicament for the treatment of glaucoma or ocular hypertension, said composition comprising brimonidine having a concentration from about 1 mM to about 4.5 mM, and timolol having a concentration from about 2 mM to about 15.8 mM, and benzalkonium chloride having a concentration of from about 150 to 250 ppm.
